(19) United States
(12) Patent Application Publication
(10) Pub. No.: US 2018/0228745 A1
(43) Pub. Date: Aug. 16, 2018

(54) SUBSTANCE SELECTED AMONG MIDODRINE, A PHARMACEUTICAL SALT AND AN ACTIVE METABOLITE THEREOF, FOR USE IN THE TREATMENT OF OBSTRUCTIVE CARDIOPATHY

(71) Applicants: UNIVERSITÉ DE BORDEAUX, Bordeaux (FR); CENTRE HOSPITALIER UNIVERSITAIRE DE BORDEAUX, Talence Cedex (FR)

(72) Inventor: Stéphane LAFITTE, Pessac (FR)

(21) Appl. No.: 15/747,890

(22) PCT Filed: Jul. 28, 2016
(86) PCT No.: PCT/EP2016/068073

§ 371 Date: Jan. 26, 2018

(30) Foreign Application Priority Data
Jul. 31, 2015 (EP) 15306251.8

(51) Int. Cl. A61K 31/165; A61K 9/00; A61K 9/08; A61P 9/02

(52) U.S. Cl. CPC A61K 31/165; A61K 9/0053; A61K 9/0029; A61K 31/138; A61P 9/02; A61K 2300/00; A61K 9/08

(57) ABSTRACT

Some embodiments are directed to a substance for use in the treatment of obstructive cardiopathy, and in particular to a substance selected among midodrine, a pharmaceutical salt, a prodrug and an active metabolite thereof, for use in the treatment of obstructive cardiopathy.

### CROSS REFERENCE TO RELATED APPLICATIONS

This application is a national phase filing under 35 C.F.R. § 371 of and claims priority to PCT Patent Application No. PCT/EP2016/068073, filed on Jul. 28, 2016, which claims the priority benefit under 35 U.S.C. § 119 of European Patent Application No. 15306251.8, filed on Jul. 31, 2015, the contents of each of which are hereby incorporated in their entireties by reference.

### BACKGROUND

Some embodiments are directed to a substance for use in the treatment of obstructive cardiopathy, such as in the medical, pharmaceutical and veterinary fields.

In the description below, the references into brackets ([ ]) refer to the listing of references situated at the end of the text.

Hypertrophic cardiomyopathy (HCM) is the most common heritable cardiovascular disorder, affecting 0.2% to 0.5% of the general population, and is a leading cause of sudden cardiac death in young athletes (Maron B J.: “Contemporary insights and strategies for risk stratification and prevention of sudden death in hypertrophic cardiomyopathy”, Circulation 2010; 121:445-56 [1]). The occurrence of hypertrophic cardiomyopathy is a significant cause of sudden unexpected cardiac death in any age group and as a cause of disabling cardiac symptoms. Younger people are likely to have a more severe form of hypertrophic cardiomyopathy.

HCM is frequently asymptomatic until sudden cardiac death, and for this reason some suggest routinely screening certain populations for this disease.

With HCM, the myocytes in the heart increase in size, which results in the thickening of the heart muscle. This increased thickness of heart muscle typically consists of asymmetric septal hypertrophy, and systolic anterior motion (SAM) of the mitral valve. In addition, the normal alignment of muscle cells is disrupted, a phenomenon known as myocardial disarray. HCM also causes disruptions of the electrical functions of the heart.

HCM is most commonly due to a mutation in one of 9 sarcomeric genes that results in a mutated protein in the sarcomere, the primary component of the myocyte (the muscle cell of the heart). These are predominantly single-point missense mutations in the genes for beta-myosin heavy chain (MHC), myosin-binding protein C, cardiac troponin T, or tropomyosin. These mutations cause myofibril and myocyte structural abnormalities and possible deficiencies in force generation.

The clinical course of HCM is variable. Many patients are asymptomatic or mildly symptomatic. The symptoms of HCM include dyspnea (shortness of breath) due to stiffening and decreased blood filling of the ventricles, exertional chest pain (sometimes known as angina) due to reduced or restricted blood flow to the coronary arteries, uncomfortable awareness of the heart beat (palpitations) due to the aforementioned ischemia, as well as disruption of the electrical system running through the abnormal heart muscle, lightheadedness, fatigue, fainting (called syncope) and sudden cardiac death. As mentioned, dyspnea is largely due to increased stiffness of the left ventricle, which impairs filling of the ventricles, but also leads to elevated pressure in the left ventricle and left atrium, causing back pressure and interstitial congestion in the lungs. Symptoms are not closely related to the presence or severity of an outflow tract obstruction. Often, symptoms mimic those of congestive heart failure (especially activity intolerance and dyspnea), but treatment of each is different.

### SUMMARY

Different treatments exist to improve symptoms of HCM. The primary goal of medications is to relieve symptoms such as chest pain, shortness of breath, and palpitations.

Beta blockers are considered as first-line agents, as they can slow down the heart rate.

For patients who cannot tolerate beta blockers or do not have good control of symptoms with beta blockers, nondihydropyridine calcium channel blockers, such as verapamil, can be used. These medications also decrease the heart rate, though their use in patients with severe outflow obstruction, elevated pulmonary artery wedge pressure and low blood pressures should be done with caution.

For patients who continue to have symptoms despite the above treatments, disopyramide can be considered for further symptom relief. Diuretics can be considered for patients with evidence of fluid overload, though cautiously used in those with evidence of obstruction. Patients who continue to have symptoms despite drug therapy can consider more invasive therapies.

In this purpose, surgical septal myectomy is an open heart operation done to relieve symptoms in patients who remain severely symptomatic despite medical therapy. It has been performed for more than 25 years. Surgical septal myectomy uniformly decreases left ventricular outflow tract obstruction and improves symptoms, and in experienced centers has a surgical mortality of less than 1%, as well as 85% success rate. However, complications of septal myectomy surgery include possible death, arrhythmias, infection, incessant bleeding, septal perforation/defect, stroke.

Another way of treatment is alcohol septal ablation, which is a percutaneous technique involving injection of alcohol into one or more septal branches of the left anterior descending artery. This is a technique with results similar to the surgical septal myectomy procedure but is less invasive, since it does not involve general anaesthesia and opening of the chest wall and pericardium (which are done in a septal myomectomy). In a select population with symptoms secondary to a high outflow tract gradient, alcohol septal ablation can reduce the symptoms of HCM. In addition, older individuals and those with other medical problems, for whom surgical myectomy would pose increased procedural risk, would likely benefit from the lesser invasive septal ablation procedure. When performed properly, an alcohol septal ablation induces a controlled heart attack, in which the portion of the interventricular septum that involves the left ventricular outflow tract is infarcted and will contract into a scar. However, which patients are best served by surgical myectomy, alcohol septal ablation, or medical therapy is an important topic and one which is intensely debated in medical scientific circles.

Another alternative treatment is the use of a pacemaker that has been advocated in a subset of individuals, in order to cause asynchronous contraction of the left ventricle. Since the pacemaker activates the interventricular septum before the left ventricular free wall, the gradient across the left ventricular outflow tract may decrease. This form of treatment has been shown to provide less relief of symptoms and less of a reduction in the left ventricular outflow tract gradient when compared to surgical myectomy. Technological advancements have also led to the development of a dual-chamber pacemaker, which is only turned on when needed (in contrast to a regular pacemaker which provides a constant stimulus). Although the dual-chamber pacemaker has shown to decrease ventricular outflow tract obstruction, experimental trials have only found few individuals with improved symptoms. Unfortunately, researchers suspect that these reports of “improved” symptoms are due to a placebo effect.

In cases that are refractory to all other forms of treatment, cardiac transplantation is one option. It is also the only treatment available for end-stage heart failure. However, transplantation must occur before the onset of symptoms such as pulmonary vessel hypertension, kidney malfunction, and thromboembolism in order for it to be successful. Studies have indicated a seven-year survival rate of 94% in patients after transplantation.

In two-third of CMH patients, CMH causes an obstruction to blood ejection, whether at rest or during effort. For those patients, resting left ventricular outflow tract obstruction (LVOTO) due to SAM is observed in 25% to 30% and, when severe CMH, may cause dyspnea, chest pain, syncope, and a predisposition to developing atrial arrhythmias (Wigle E D, Sasson Z, Henderson M A, et al.: “Hypertrophic cardiomyopathy. The importance of the site and the extent of hypertrophy. A review', Prog Cardiovasc Dis 1985; 28:1-83 [2]). For these patients, all the common efforts of routine life are rendered unbearable.

Insights into the pathophysiology of LVOTO have recently been provided by exercise echocardiography, which can quantify the gradient during or after exercise (Maron M S, Olivotto I, Zenovich A G, et al. Hypertrophic cardiomyopathy is predominantly a disease of left ventricular outflow tract obstruction. Circulation 2006; 114:2232-9 [3]; Shah J S, Esteban M T, Thaman R, et al. Prevalence of exercise-induced left ventricular outflow tract obstruction in symptomatic patients with non-obstructive hypertrophic cardiomyopathy. Heart 2008; 94:1288-94 [4]; Joshi S, Patel U K, Yao S S, et al. Standing and exercise Doppler echocardiography in obstructive hypertrophic cardiomyopathy: the range of gradients with upright activity. J Am Soc Echocardiogr 2010; 24:75-82 [5]; Argulian E, Chaudhry F A. Stress testing in patients with hypertrophic cardiomyopathy. Prog Cardiovasc Dis 2012; 54:477-82 [6]). Although the Venturi effect was believed to be responsible for SAM ([4]; Maron B J, Gottdiener J S, Roberts W C, Henry W L, Savage D D, Epstein S E. Left ventricular outflow tract obstruction due to systolic anterior motion of the anterior mitral leaflet in patients with concentric left ventricular hypertrophy. Circulation 1978; 57:527-33 [7]; Maron B J, Harding A M, Spirito P, Roberts W C, Waller B F. Systolic anterior motion of the posterior mitral leaflet: a previously unrecognized cause of dynamic subaortic obstruction in patients with hypertrophic cardiomyopathy. Circulation 1983; 68:282-93 [8]), the most recent evidence for LV obstruction in HCM patients favors the flow drag mechanism causing the mitral valve to be pushed against the septum (Gersh B J, Maron B J, Bonow R O, et al. 2011 ACCF/AHA guideline for the diagnosis and treatment of hypertrophic cardiomyopathy: executive summary. A report of the American College of Cardiology Foundation/American Heart Association Task Force on Practice Guidelines. J Am Coll Cardiol 2011; 58:2703-38 [9]). The mechanism of obstruction is probably also related to other alterations produced by HCM. Experimental and observational data suggest that anterior displacement of the papillary muscles and submitral apparatus is necessary to create sufficient leaflet slack to allow for anterior motion of the mitral leaflet (Cavalcante J L, Barboza J S, Lever H M. Diversity of mitral valve abnormalities in obstructive hypertrophic cardiomyopathy. Prog Cardiovasc Dis 2012; 54:517-22 [10]; Levine R A, Vlahakes G J, Lefebvre X, et al. Papillary muscle displacement causes systolic anterior motion of the mitral valve. Experimental validation and insights into the mechanism of subaortic obstruction. Circulation 1995; 91:1189-95 [11]). Nevertheless, the major potential effects of ventricular loading and myocardial contractility must also be considered. These effects may be exerted both in early systole, for which flow, drag, and pushing force of flow are the dominant hydrodynamic forces for SAM, and at midsystole, for which the displacing force is more prominent ([5]). Hence, small variations in preload, afterload, or contractility, such as produced by exertion, may lead to large changes in gradient, usually explaining the amplification of obstruction from rest to exercise or from exercise to recovery.

To date, drug treatments such as beta-blockers or calcium channel blockers are moderately effective and sometimes associated with unacceptable adverse effects in CMH patients with obstruction to blood ejection, whether at rest or during effort. In this respect, dihydropyridine calcium channel blockers are traditionally avoided in patients with evidence of obstruction.

Unfortunately, this problem of obstruction to blood ejection can also affect other heart conditions, even without associated evident hypertrophy, with the same symptoms as those of CMH.

Thus, it may be beneficial to provide alternative, more efficient and less toxic treatments of heart diseases with obstruction to blood ejection, in particular CMH with obstruction to blood ejection. Some of the disclosed embodiments are directed to fulfilling these and other needs.

After important researches on series of obstructive heart diseases, the present Applicants are the first ones to have experimented and observed that midodrine may be very efficient to improve or enhance the symptoms of obstruction to blood ejection, while being less toxic on these pathologies than traditional treatments as beta-blockers or calcium channel blockers.

The Applicant surprisingly found that midodrine improves exercise breathlessness, exercise chest pain, exercise discomfort and exercise dizziness in patients with an obstructive heart disease.

In this purpose, the Applicant demonstrated an enhancement or improvement of the venous return, an immediate decrease of the intraventricular obstruction and a decrease of the hyperkinetic state in patients due to administration of midodrine. Surprisingly, the Applicants demonstrated that the immediate increase of venous return allowed decreasing the intraventricular obstruction. The Applicants also surprisingly observed an inverted phenomenon (relapse and increase of obstruction) after few hours stop of the administration of midodrine, hypothesizing the major role of midodrine in balancing the total blood pool from the veins towards the heart.

Accordingly, in a first aspect, some embodiments are directed to a substance selected among midodrine, a pharmaceutical salt, a prodrug or an active metabolite thereof, for use in the treatment of obstructive cardiopathy.

“Midodrine” refers herein to an ethanomaline derivative having the following formula (I):

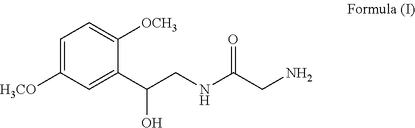 [SMILES: COc1ccc(OC)c(C(O)CNC(=O)CN)c1]

It may be designated under its chemical name 2-amino-N-[2-(2,5-dimethoxyphenyl)-2-hydroxyethyl]acetamide hydrochloride. Its CAS Number is 3092-17-9. Midodrine is a prodrug which forms an active metabolite, desglymidodrine, which has a selective sympathomimetic effect on peripheral alpha-adrenergic receptors and exerts its actions via activation of the alpha-adrenergic receptors of the arteriolar and venous vasculature, producing an increase in vascular tone and elevation of blood pressure. Administration of midodrine results in vasoconstriction of veins at first, thereby reducing the venous pool, and then, in a second time, of arteries. The Applicants hypothesize that this mechanism may be implied in the biological effect of midodrine on the improvement of the symptoms of obstructive cardiopathy observed after administration of midodrine to the patients.

Midodrine may also refer to any alpha-1-adrenergic receptor agonist substance, including midodrine, norepinephrine, dopamine, ephedrine, phenylpropanolamine, methoxamine, phenylephrine, and noradrenaline, or a pharmaceutical salt thereof. Examples of alpha-1-adrenergic receptor agonist substances that may be used in some embodiments are disclosed in Goodman and Gilman's the pharmacological basis of therapeutics, eleventh edition, chapter 10, pp 271-295, 2006 ([12]).

Midodrine was approved in the United States by the Food and Drug Administration (FDA) in 1996 for the treatment of dysautonomia and orthostatic hypotension.

Dysautonomia (or autonomic dysfunction, autonomic neuropathy) is an umbrella term for various conditions in which the autonomic nervous system (ANS) malfunctions. Dysautonomia is a type of neuropathy affecting the nerves that carry information from the brain and spinal cord to the heart, bladder, intestines, sweat glands, pupils, and blood vessels.

Orthostatic hypotension, also known as postural hypotension, orthostasis, and colloquially as head rush or dizzy spell, is a form of low blood pressure in which a person's blood pressure falls when suddenly standing up or stretching. In medical terms, it is defined as a fall in systolic blood pressure of at least 20 mmHg or diastolic blood pressure of at least 10 mmHg when a person assumes a standing position.

The term “pharmaceutical salt” is meant to include any pharmaceutically acceptable salt, the substance that are prepared with relatively nontoxic acids or bases, depending on the particular substituents found on the compounds described herein. The pharmaceutical salt includes any salt suitable to be administered to humans or animals. Examples of non toxic pharmaceutically acceptable salts may include hydrochloride, sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, hydroxybutylate, glycolate, malate, tartrate, methanesulfonate, propanesulfonate, naphthalene-1-sulfonate, naphthalene-2-sulfonate and mandelate. Pharmaceutically acceptable salt of midodrine may be midodrine hydrochloride, for example Gutron™ (Takeda).

“Active metabolite” refers herein to any therapeutically active metabolite released by activation of the prodrug, i.e. the alpha-1-adrenergic receptor agonist substance within the human body by an enzymatic hydrolysis. It may advantageously refer to desglymidodrine, which is the active metabolite of midodrine. Advantageously, desglymidodrine acts by a stimulation of alpha-1 adrenergic receptors of the arteriolar and venous vasculature, producing an increase in vascular tone and elevation of blood pressure; it diffuses poorly across the blood-brain barrier, and is therefore not associated with effects on the central nervous system.

«Prodrug » refers herein to any precursor compound which may be administered in a pharmacologically inactive form, and which is likely to be converted in an active form through a normal metabolic process in physiological conditions. The active form may be midodrine or an active metabolite of midodrine, as desglymidodrine.

“Obstructive cardiopathy”, also named “obstructive cardiomyopathy”, refers herein to any heart disease having an intraventricular obstruction. In other words, it may refer to an obstructive cardiac disease. For example, the intraventricular obstruction may occur at rest, i.e. when oxygen consumption of the body is stable, and/or during exercise, i.e. when oxygen consumption increases due to the realization of a movement by the body and/or throughout the recovery phase after exercise, meaning within the next 5 to 10 minutes after the exercise has stopped. Exercise may be for example, a situation when a body assumes a standing position, or walking, or climbing stairs, or lifting a load. The intraventricular obstruction corresponds to the existence of a pressure gradient observable by Doppler echocardiography, especially of left ventricle structure and function, at rest and/or during exercise. The gradient may be above 30 mmHg at rest, for example strictly above 35 mmHg, or above 40 mmHg, or above 50 mmHg, or above 60 mmHg, or above 70 mmHg, or above 100 mmHg, or for example of about 130 mmHg. The gradient may be above 50 mmHg during exercise, for example above 55 mmHg, or above 60 mmHg, or above 80 mmHg, or above 100 mmHg, or above 110 mmHg, or above 150 mmHg, or for example of about 180 mmHg. Resting echocardiography may be a resting 2-dimensional (2D) echocardiography for example performed according to American Society of Echocardiography guidelines (Lang R M, Bierig M, Devereux R B, et al. Recommendations for chamber quantification: a report from the American Society of Echocardiography's Guidelines and Standards Committee and the Chamber Quantification Writing Group. J Am Soc Echocardiogr 2005; 18: 1440-63 [13]; Gottdiener J S, Bednarz J, Devereux R, et al. American Society of Echocardiography recommendations for use of echocardiography in clinical trials. J Am Soc Echocardiogr 2004; 17:1086-119 [14]), with ultrasound recordings by an experienced (level 3) operator (Quinones M A, Douglas P S, Foster E, et al. ACC/AHA clinical competence statement on echocardiography: a report of the American College of Cardiology/American Heart Association/American

College of Physicians-American Society of Internal Medicine Task Force on Clinical Competence. J Am Coll Cardiol 2003; 41:687-708 [15]). Exercise echocardiography may be performed for example as bicycle exertion in a semisuspine position (50°) to enable simultaneous transthoracic echocardiography during exercise.

The obstructive cardiopathy may be an obstructive cardiomyopathy. It may be selected among hypertrophic cardiomyopathy, hypertensive cardiopathy, a primary hyperkinetic function, a malposition of papillary muscles and a situation with primary hyperkinesia of left ventricle in non hypertrophic cardiomyopathy.

“Treatment” refers herein to any improvement of at least one symptom of the obstructive cardiopathy, or to the recovery of the obstructive cardiopathy, or to the prevention of the symptoms of the obstructive cardiopathy. The recovery of the obstructive cardiopathy may be a decrease or a disappearance of the obstruction. The symptoms of the obstructive cardiopathy may be anyone of those of the New York Heart Association (NYHA) Functional Classification. The at least one symptom that may be improved may be selected among breathlessness, especially exercise breathlessness, chest pain, especially exercise chest pain, discomfort, especially exercise and dizziness, especially exercise dizziness. The improvement of at least one symptom may refer to total or partial disappearance of at least one symptom, which may be transient or definitive, at rest and/or during exercise. The improvement of at least one symptom may refer for example to a decrease of NYHA class according to NYHA Functional Classification.

Advantageously, the substance may increase the venous return of blood to heart, thus decreasing the hyperkinetic state of the cardiopathy; i.e. decreasing the heart contraction and/or decreasing heart flow rate, and/or decreasing circulatory rate. Advantageously, the substance may increase loading conditions, i.e. afterload and preload.

Advantageously, the substance may be administered to a patient in need thereof to a pharmaceutically acceptable and efficient dose for the treatment of obstructive disease.

Advantageously, it may be a dose increasing the venous return of blood to heart. Advantageously, the substance is administrated from one to eight times per 24 hours, i.e. per day. For example, the dose(s) may be administrated once per day, or twice per day, or three times per day, or four times per day, or five times per day, or more until eight times per day. For example, it may be administrated from one to three, or to one to four times per day, i.e. per 24 hours. For example, the total dose administrated per day may be included of from 6 mg to 45 mg per day, i.e. per 24 hours. For example, the substance may be administered from 1 to 3 dose(s) of 2.5 mg each of the substance, from 1 to 3 times per day, or from 1 to 4 times per day. For example, the dosing regimen may be advantageously selected to maintain the number of drug intakes per day (i.e. per 24 hours) when searching for the more adapted dosing regimen for a patient, while increasing the quantity of administered substance per drug intake. It may correspond to an administration of from about 0.1 mg of the substance/kg of patient, to about 0.75 mg of the substance/kg of patient, per day. In the particular case where the patient is an infant, one of ordinary skill in the art would adapt the dosing regimen according to his general technical knowledge.

According to some embodiments, the substance may be administered for sufficient time for the treatment of obstructive cardiopathy as defined above. In this purpose, the substance may be administered for one month, or for two months, or for three months, or for six months, or for at least six months, or for the patient's lifetime.

The substance may be in any suitable form for an administration to a human or an animal, especially in the form of a medicament. In other words, the patient may be a human or an animal. The animal may be selected from the group including felids, for example cats, canids, for example dogs, cervids, equids, mustelids, procyonids, viverrids and ursids. More particularly, the animal may be a domestic animal selected among cats, dogs, hamsters, rabbits, guinea pigs and ferrets. For example, the substance may be administered for the treatment of hypertrophic cardiomyopathy to a cat or a dog.

Cats may be of any breed, and of any age, for example from a few months to 15 years old cats. It may be for example a European wildcat, a British cat, a Maine coon cat, an Egyptian Mauscat , a Norvegian cat, a Persian cat, a Ragdoll cat, a Rex Cornish cat, a Rex Devon cat or a Sphynx cat.

Dogs may be of any breed, and of any age, for example 3 years old dogs. It may be for example a Boston Terrier dog, a Airedale Terrier dog, a Akita Inu dog, an Alaskan Malamute dog, an American Bulldog, an American Cocker Spaniel dog, a Jack Russell Terrier dog, a Japanese Chin dog, a Japanese Spitz dog, an Old Danish Pointer dog, an Old German Shepherd Dog.

Administration may be carried out directly, i.e. pure or substantially pure, or after mixing of the substance with a pharmaceutically acceptable carrier and/or medium.

Administration of the substance may be carried out either simultaneously, separately or sequentially with at least one another active compound(s) selected in the group including beta-blockers, for example propranolol, bisoprolol, carvedilol and nadolol, calcium inhibitors, for example verapamil, antiarythmic agents, for example amiodarone, and anticoagulant agents, for example rivaroxaban. The substance and the at least one another active compound(s) mixture may be administered in a relative amount of 0:1 to 1:0, for example 1:1. The other active compound may be used for the treatment of obstructive cardiopathy, or for another pathology existing together with the obstructive cardiopathy, for example an orthostatic hypotension, or a rhythm disorders such as atrial fibrillation.

In one embodiment, the administration may be an oral administration. In this embodiment, it may be a buccal or a sublingual administration. It may for example be in the form selected from the group including a liquid formulation, an oral effervescent dosage form, an oral powder, a pill, a multiparticule system, an orodispersible dosage form, a solution, a syrup, a suspension, an emulsion and oral drops. When the medicament is in the form of an oral effervescent dosage form, it may be in a form selected from the group including tablets, granules, powders. When the medicament is the form of an oral powder or a multiparticulate system, it may be in a form selected from the group including beads, granules, mini tablets and micro granules. When the medicament is the form of an orodispersible dosage form, it may be in a form selected from the group including orodispersible tablets, lyophilized wafers, thin films, a chewable tablet, a tablet and a capsule, a medical chewing gum. According to some embodiments, when the medicament is for buccal and sublingual routes, it may be selected from the group including buccal or sublingual tablets, muco adhesive preparation, oro-mucosal drops and sprays.

In another embodiment, the administration is a parenteral administration. The parenteral administration may be selected from the group including intravenous administration, intramuscular administration and subcutaneous administration. In those cases, the substance may be in the form of an injectable solution.

In another embodiment, the administration may be a transdermal or a transmucosal administration. When the medicament is for topical-transdermal administration, it may be selected from the group including ointments, cream, gel, lotion, patch and foam. It may also be a medicament for nasal administration, for example selected from the group including nasal drops, nasal spray, nasal powder.

In another embodiment, the administration may be a rectal administration, for example suppository or hard gelatin capsule.

One of ordinary skill in the art understands clearly that the term “form” as used herein refers to the pharmaceutical formulation, including veterinary formulation, of the medicament for its practical use. For example, the medicament may be in a form selected from the group including an injectable form (for example as Avlocardyl®5 mg/ml), syrup (for example as Efferalgan®3%), oral suspension (for example as Efferalgan® 3%), a pellet (for example as Dafalgan®1 g), powder (for example as Doliprane®100 mg), granules (for example as Zoltum®10 mg), spray, transdermal patch (for example as Cordipatch®5 mg/24 h) or local form (cream, lotion, collyrium) (for example as Dermoval creme®, as Betneval®lotion and as Chibroxine® collyre respectively).

In these examples, the substance, for example midodrine, may be added or may replace the active ingredient(s) of said medicaments.

The pharmaceutically acceptable carrier may be any know suitable pharmaceutically and veterinary carrier used for the administration of a substance to a human or to an animal, depending on the subject. For example, this carrier may be one or more carrier(s) selected from the group including for example the monomethoxy-polyethyleneglycol (for example as in Viraferonpeg®), Liposome (for example as in Ambizome®), magnesium stearate (E572), talc (E553b), Silicon dioxide (E551), microcrystalline cellulose (E460) and maize starch. Preferably, the carrier includes magnesium stearate (E572), talc (E553b), Silicon dioxide (E551), microcrystalline cellulose (E460) and maize starch.

The medium may be any know medium used for the administration of a substance to a human or to an animal. For example, this medium may be selected from the group including for example cremophor (for example as in Sandimmun®) or cellulosis (for example as in Avlocardyl® LP160 mg). The pharmaceutical form of the drug is selected with regard to the human or animal to be treated. For example, for a child or a baby, a syrup or an injection is preferred. Administration may be carried out with a weight graduated pipette. In the case where the patient is an animal, the pharmaceutical form of the drug may be for example an oral form that may be selected among drinkable solutions, dragees, capsules, gel, emulsions, pastes, suspensions, sublingual film, soft or hard tablets, soft tablets to be chewed, film-coated tablets, effervescent tablets, soluble tablet, dispersible tablets, tablets orodispersible, soft or hard capsules, soft capsules to chew, pellets or granules to be dissolved or dispersed on food, in water of drink, a presentation out of sachets or a pot with pod, powders to be dissolved or dispersed on food, in the drink water, syrups, functional food, liquids to be dispersed on food and of hydrogels, or any other suitable form. According to a particular aspect of some embodiments, the substance may be managed as a component of a complete feeding stuff for animals, or a treat. Preferably, these different forms may present a palatable aspect for the animal, so that the animal may ingest easily the drug.

In a second aspect, some embodiments provide a method of treating a subject suffering from an obstructive cardiopathy as defined above, including a step of administering to said subject a substance as defined above.

In another aspect, some embodiments provide the use of a substance as defined above for the manufacture of a medicament for the treatment of an obstructive cardiopathy as defined above.

Some embodiments are further illustrated by the following examples with regard to the annexed drawings that should not be construed as limiting.

### BRIEF DESCRIPTION OF THE FIGURES

FIG. 1: represents a 2D echocardiography of left ventricle revealing a bulging septum of thickness: 12-13 mm with incomplete systolic anterior motion of the mitral valve (SAM) at rest. No significant rest obstruction was observed.

FIG. 2: represents a Doppler record of left ventricle systolic flow revealing a severe obstruction up to 100 mmHg after exercise.

FIG. 3: represents a 2D echocardiography of left ventricle during exercise with the appearance of a complete SAM.

FIG. 4: represents a 2D echocardiography at rest in which systolic left ventricular volume in this HCM patients was minimal at least virtual with a physical contact between septal and lateral walls.

FIG. 5: represents a Doppler echocardiography at rest with a significant obstruction above of 80 mmHg.

FIG. 6: represents a Doppler echocardiography exercise. The obstruction was increased with a maximum gradient of 118 mmHg.

FIG. 7: represents an exercise stress Doppler echocardiography during chest pain experienced in everyday life obtained after a short distance walk (less than 20 meters). An intra ventricular obstruction up to 145 mmHg is recorded.

FIG. 8: represents an exercise stress Doppler echocardiography during chest pain experienced in everyday life obtained after a short distance walk (less than 20 meters). The parietal contact between the septal and lateral walls was increased.

FIG. 9: represents a Doppler echocardiography during a leg-raise test, that reveals an immediate reduction in the intraventricular obstruction, measured at 80 mmHg.

FIG. 10: represents an echocardiographic follow-up examination under treatment revealing a significantly-reduced maximum gradient of the left intraventricular obstruction at rest, down to 30 mmHg in comparison with 80 mmHg recorded before treatment initiation.

FIG. 11: represents an echocardiographic follow-up examination under treatment revealing a significantly-reduced maximum gradient of the left intraventricular obstruction at 60 mmHg post-exercise compared to 140 mmHg before treatment.

FIG. 12: represents an echocardiographic revealing an hypertrophic cardiomyopathy (HCM), obstructive at rest, with a gradient superior to 100 mmHg, along with high-grade mitral insufficiency in a hyperkinetic left ventricle.

FIG. 13: represents a Doppler echocardiography during a leg-raise test to increase venous return under echocardiographic control, which instantly reduced the mitral insufficiency to a mild grade and the obstruction to 40 mmHg.

FIG. 14: represents a Doppler echocardiography at rest, showing absence of obstruction (maximum gradient of 18 mmHg), a mitral insufficiency of a milder degree, and pulmonary pressures normal (32 mmHg).

FIG. 15: represents an immediate post-exercise echocardiography Doppler after walking and stair stress tests (50 meters walking and 30 steps), revealing similar characteristics as those of the resting echocardiography (absence of obstruction with a maximum gradient of 18 mmHg).

### EXAMPLES

### Example 1

### Description of a First Clinical Case

The first patient was 45 years old and admitted to general cardiology at Bordeaux University Hospital (CHU Bordeaux) for orthostatic hypotension following a previous diagnosis of obstructive cardiomyopathy (confirmed by a MYH7 gene mutation). Treatment with Nadolol at 80 mg/day was unable to prevent several lipothymic and syncopal episodes. He also complained of significant discomfort when walking, classed as New York Heart Association (NYHA) dyspnea 2-3.

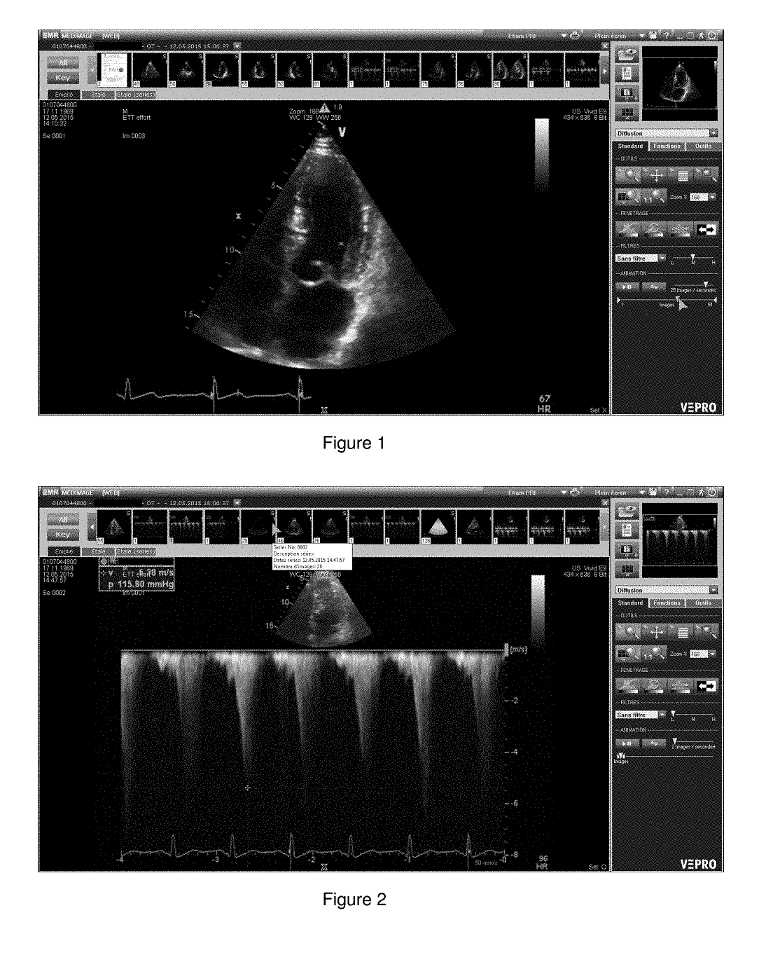

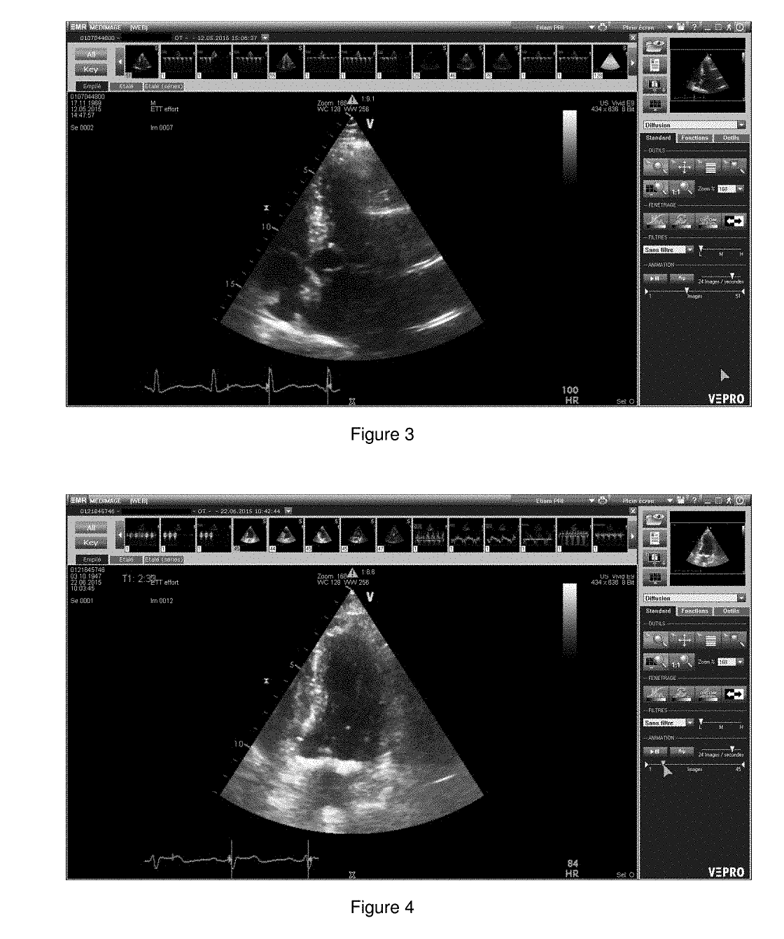

Echocardiography revealed bulging septum (thickness: 12-13 mm) with incomplete systolic anterior motion of the mitral valve (SAM) at rest (FIG. 1). There was no intra-ventricular obstruction at rest, yet during exercise it was recorded at 50 mmHg gradient, which increased during early recovery to 100 mmHg (FIGS. 2 and 3). Concurrently, the patient presented clinically with his usual symptoms of faintness and dizziness.

Tilt test was performed during his hospitalization, revealing clear orthostatic hypotension after NATISPRAY administration with decreasing blood pressure from 124/80 mmHg to 80/48 mmHg, also accompanied by faintness, hot flashes, extreme paleness, and blackout.

Monitoring with continuous ECG during hospitalization revealed no heart rhythm or conduction disorders.

Considering the patient's orthostatic hypotension, a treatment with midodrine hydrochloride (Gutron™) was initiated at a dose of 3 2.5-mg tablets per day. This drug produces a direct and selective sympathomimetic effect on peripheral alpha-adrenergic receptors, resulting in vasoconstriction of first the veins (reducing the venous pool) then the arteries. This prevents orthostatic disorders, increases peripheral resistance, and causes a rise in blood pressure.

After 1 month of treatment, faintness did not recur. In addition, the patient reported less shortness of breath during exercise (NYHA severe 2 to severe 1).

Following 6 weeks of treatment, the patient exhibited no longer symptoms or limitations during exercise. Echocardiography demonstrated a total disappearance of obstruction at rest, during exercise or recovery.

We therefore conclude that midodrine hydrochloride, initially prescribed for orthostatic hypotension in this patient with intra-ventricular obstruction, also resolved shortness of breath during exercise. We believe this to be the result of an improvement in cardiac filling, hence resulting in left ventricular cavity dilatation and reduction of the intra-ventricular obstruction responsible for the symptoms.

Immediate improvement of symptoms obtained at the treatment beginning was still present after one year. No additive improvement was observed after the first control neither other modification of the obstruction.

### Example 2

### Description of a Second Clinical Case

The second patient, of female gender, suffered from familial hypertrophic cardiomyopathy (HCM) and was admitted for disabling chest pain during exercise accompanied by shortness of breath during even the mildest activity (walking for less than 10 m).

The complete work-up of her cardiomyopathy performed 6 months earlier had revealed:

Echocardiography: septal obstructive predominant hypertrophic cardiomyopathy (HCM) at 14 mm thickness in the septum, subaortic obstruction with a maximum gradient of 88 mmHg, SAM, Grade 2 mitral insufficiency, preserved left ventricular ejection fraction, and absence of aortic valvulopathy.

Exercise stress echo test: exercise stress ultrasound with no medication set at 92% of the theoretical maximum heart rate and being clinically, electrically, and echographycally negative for ischemia. Obstruction at rest, resolving during exercise and increasing during recovery (90 mmHg), absence of arrhythmia, appropriate blood pressure profile.

Cardiac magnetic resonance imaging (MRI): obstructive HCM with slight SAM, and preserved segmental and global left ventricular function.

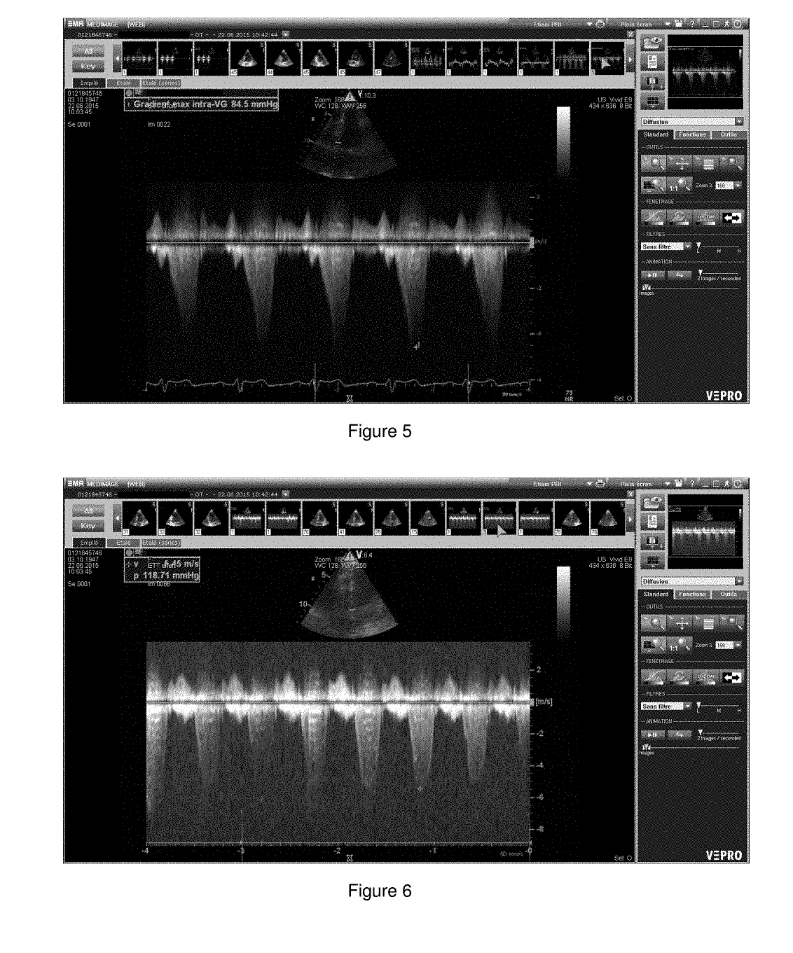

During her hospital stay this time, she underwent an exercise stress echocardiography. Resting echocardiography revealed an obstructive HCM with a gradient of 80 mmHg (FIGS. 4 and 5), along with Grade 2 mitral insufficiency with no increase in filling pressures, and her pulmonary pressures were estimated at 35 mmHg.

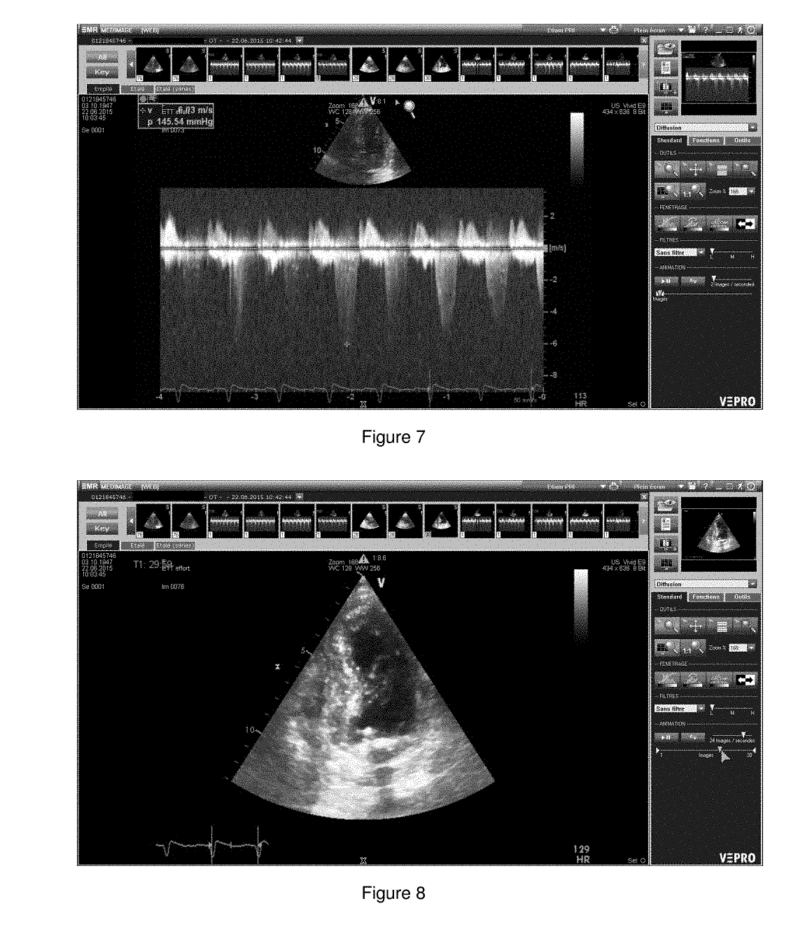

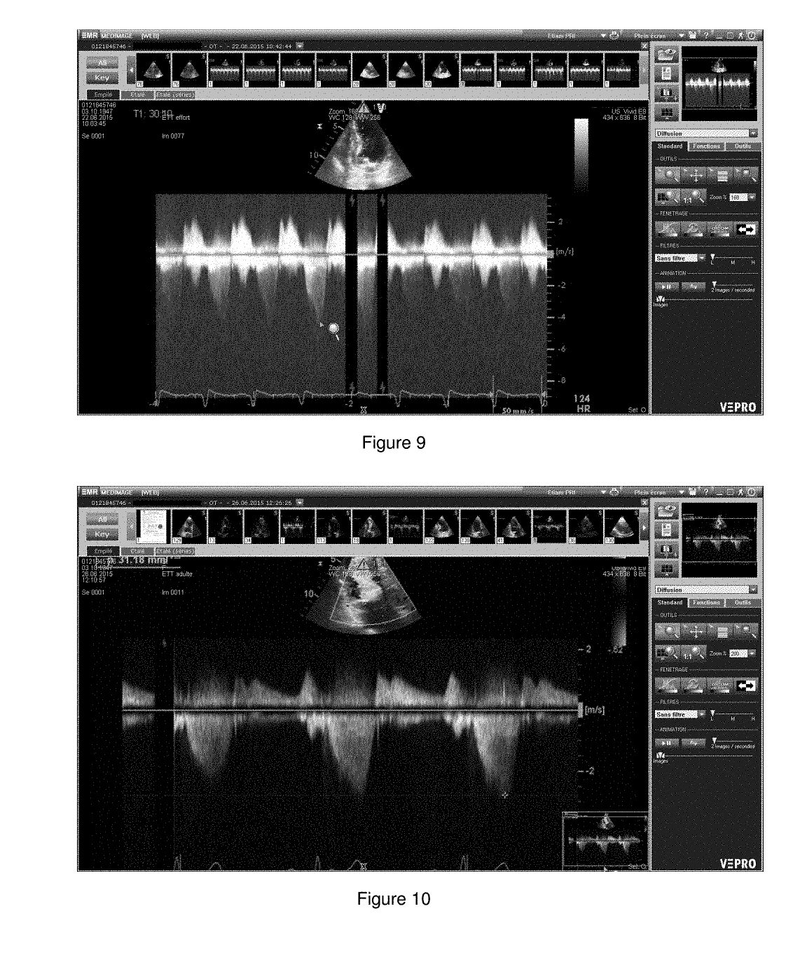

During exercise, performed on a bicycle at 75 watts and 90% of theoretical maximum heart rate, her blood pressure adapted well and no electrical modification or degradation of the left ventricular function was observed. The obstruction was seen to increase, with a maximum gradient of 120 mmHg (FIG. 6), yet the patient exhibited few symptoms, presenting solely with a Grade 1-2 dyspnea with no reproduction of pain. It was also decided to attempt to reproduce the chest pain experienced in everyday life, i.e., when walking, which caused debilitating and violent pain, with a maximum gradient of 145 mmHg (FIG. 7) and featuring significant parietal contact between the septal and lateral walls (columns) (FIG. 8). On performing a leg-raise test, echocardiography revealed an immediate reduction in the intraventricular obstruction, measured at 80 mmHg (FIG. 9). ECG tracings recorded throughout this test revealed no change indicative of myocardial ischemia.

Coronary angiography was performed to investigate suspicious pain but revealed no anomalies.

Given the leg-raise test results, a treatment aimed at increasing cardiac filling was considered. The test was also repeated with compression stockings, which had no beneficial effect on the patient's comfort.

Considering the improvement achieved by administering Gutron™ to the first patient and the debilitating nature of this patient's chest pain, a treatment attempt was made with Gutron™, administered initially in the form of 2.5 mg tablets. The patient received first one 2.5 mg tablet three times per day and then two 2.5 mg tablets three times per day. An increase of the effect has been observed. Within 48 hours of initiating treatment, the patient became asymptomatic, relieved of chest pain, and exhibited no more shortness of breath during exercise (tested by means of two flights of stairs ascension while being monitored medically, with no discomfort experienced).

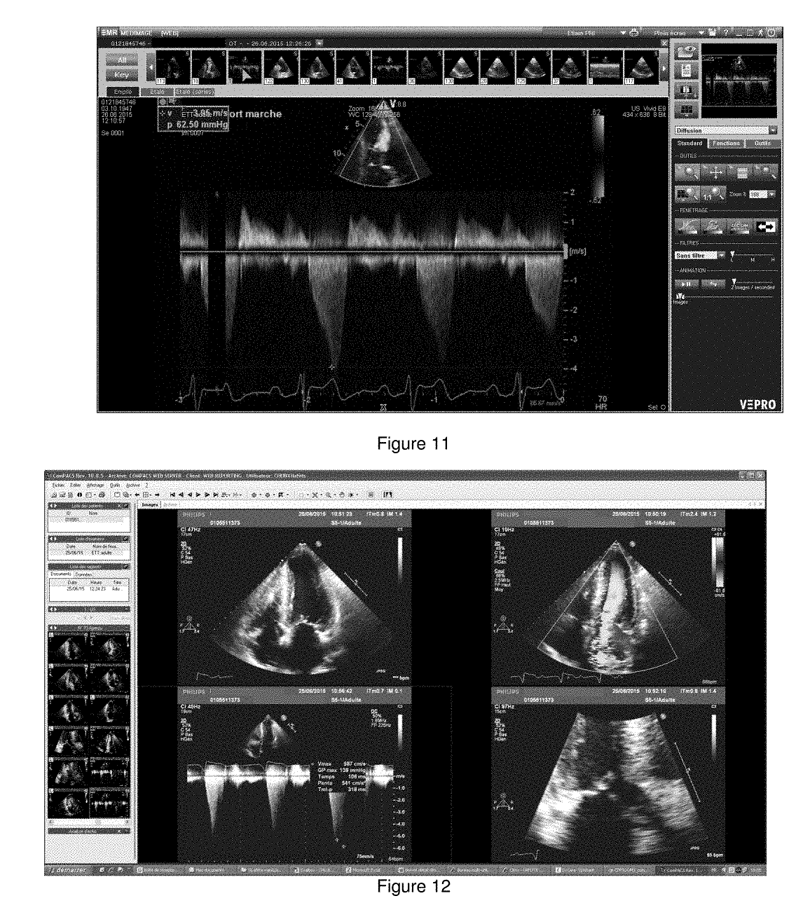

Echocardiographic follow-up examination under treatment revealed a significantly-reduced maximum gradient of the left intraventricular obstruction at rest, down to 30 mmHg in comparison with 80 mmHg recorded before treatment initiation (FIG. 10), and 60 mmHg post-exercise compared to 140 mmHg before treatment (FIG. 11). It is believed that the left ventricular filling was improved by this vasoactive treatment, which also caused a decrease in the intraventricular obstruction.

The patient was discharged. Two weeks following her departure, a follow-up interview was conducted by telephone, in which the patient reported continued improvement with near-absence of chest pain, which no longer occurred every day, nor limited her daily activities. She evaluated her quality of life to have improved from 2/10 to 7/10, and reported the complete absence of shortness of breath when she exercises.

The patient was then asked to stop the midodrine hydrochloride (Gutron™) for 2 days. After only 4 hours, she starting feeling limited during light exertion with reappearance of chest pains and shortness of breath. Immediate re-absorption of Gutron™ released the symptoms.

### Example 3

### Description of a Third Clinical Case

This 66-year-old patient had been diagnosed many years previously with hypertrophic cardiomyopathy (HCM) and was fitted with a dual-chamber pace maker due to a paroxysmal atrioventricular block. He also presented an early-stage chronic obstructive bronchopneumopathy, caused by smoking, limiting the possibility of beta-blocker prescription. His medical history notably included the sudden death of a sister at 35 years old, potentially indicating a family history of HCM.

The patient complained of dyspnea in everyday activities, classed as NYHA severe 2 (walking 10 meters and stopping due to shortness of breath). He was treated with 2.5 mg carvedilol, 200 mg amiodarone, and 20 mg rivaroxaban.

He was admitted to the emergency room for acute respiratory distress secondary to an acute pulmonary edema. Echocardiography revealed a HCM, obstructive at rest, with a gradient superior to 100 mmHg, along with high-grade mitral insufficiency in a hyperkinetic left ventricle (FIG. 12). With the appropriate medical treatment, he was able to recover a stable hemodynamic status within 3 days, and his cardiac insufficiency symptoms were reduced. His pace maker was also updated, and a coronary angiography was performed, coming back normal.

However, 1 week later, a further echocardiography was performed that revealed functional characteristics very similar to those observed on the first echocardiography, with a still-present obstruction at 90 mmHg, high-grade mitral insufficiency, and pulmonary pressures of approximately 55 mmHg, indicating a fragile hemodynamic status.

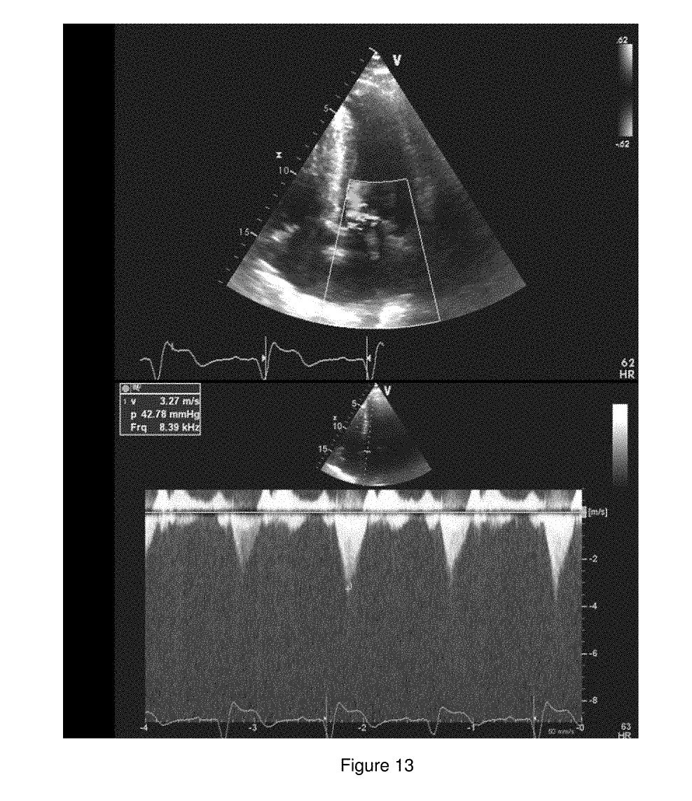

It was then decided to perform the leg-raise test to increase venous return under echocardiographic control, which instantly reduced the mitral insufficiency to a mild grade and the obstruction to 40 mmHg (FIG. 13). Given the absence of immediate treatment options, the patient was started on a course of midodrine hydrochloride (Gutron™) at a dose of 8 tablets of 2.5 mg per day, due to his low blood pressure.

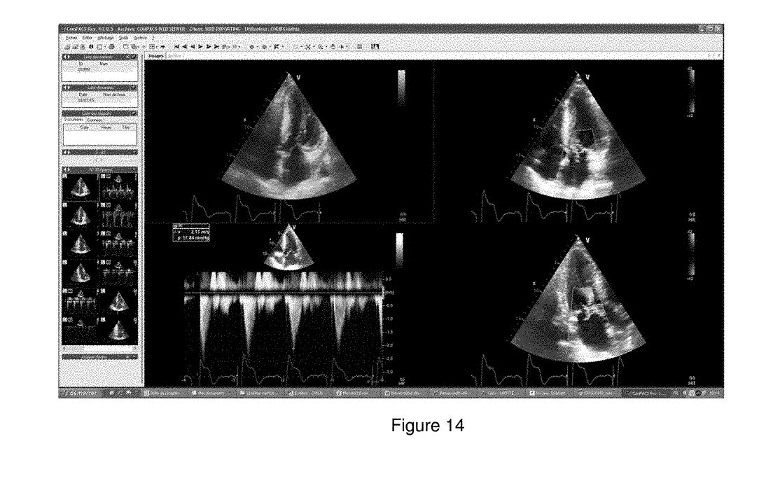

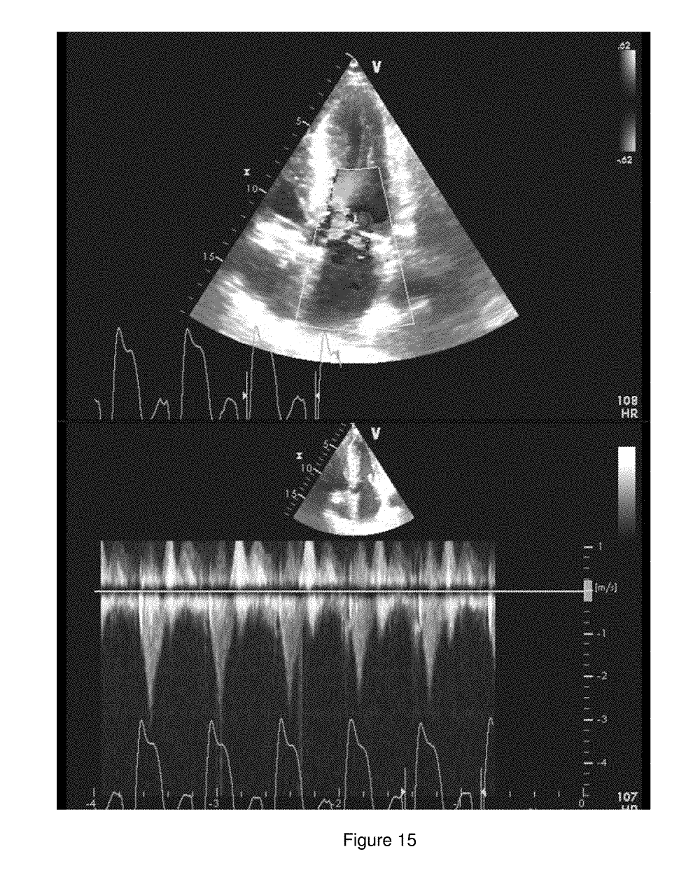

Following 24 hours of treatment, the patient was again evaluated by echocardiography. At rest, the obstruction was entirely absent (maximum gradient of 18 mmHg), the mitral insufficiency of a milder degree, and pulmonary pressures normal (32 mmHg) (FIG. 14). The patient was asked to perform walking and stair stress tests (50 meters walking and 30 steps), which posed him no problem whatsoever, and he experienced no shortness of breath. The immediate post-exercise echocardiography (heart rate: 100 bpm) revealed similar characteristics as those of the resting echocardiography (FIG. 15).

The patient was followed up 1 week following treatment initiation, at which point he was still asymptomatic. Echocardiography confirmed his improved hemodynamic profile, with a total absence of the gradient and mitral insufficiency.

Immediate improvement of symptoms obtained at the treatment beginning was still present after one year. No additional improvement was observed after the first control neither other modification of the obstruction.

### Example 4

### Clinical Study

A prospective monocentric, randomized, placebo controlled in parallel, double-blind clinical trial is conducted at the University Hospital Center of Bordeaux. Forty patients with obstruction to blood ejection, whether at rest or during effort, with persistence symptoms despite optimal treatment, are randomized as follows:

Arm 1 (20 patients): Midodrine, 5 mg, 3 times a day, 30 days

Arm 2 (20 patients): Placebo, 5 mg, 3 times a day, 30 days

A walk distance test and an exercise echocardiography at day 15 are performed and compared to the evaluation before the treatment start (day 0).

Dosage is then adapted, depending on symptoms release and walk distance test results for another 15 days period. A new evaluation is performed at 30 days including chlorydrate of midodrine tolerance, walk distance test and exercise echocardiography.

Data from each analysis time are compared between the 2 groups and from patient to patient evolution.

The main objective of the study is to assess the efficacy of 15 and 30 days midodrine treatment taken as described above on improving the distance covered during a 6 minutes walk test (6MWT).

The six-minute walk test (6MWT) measures the distance an individual is able to walk over a total of six minutes on a hard, flat surface. The goal is for the individual to walk as far as possible in six minutes. A comparison between Arm 1 and Arm 2 is done.

Moreover, the symptoms of HCM including dyspnea, exertional chest pain, uncomfortable awareness of the heart beat are assessed.

Rest and exercise echocardiography is performed before (day 0), at 15 and 30 days of treatments in order to measure the impact of treatment upon left ventricular obstruction, evolution of NYHA functional class, quality of life tests and treatment tolerance in the two groups.

### Example 5

### Description of a Fourth Clinical Case

This 53 years old patient suffered of hypertrophic cardiomyopathy. He presented a septum wall measured at 22 mm, had a treatment based on Nadolol (120 mg/day), and suffered from exercise short breathness (NYHA 2) especially on moderate daylife activities.

An echocardiography revealed a rest obstruction at 70 mmHg, increasing to 80 mmHg during walking test.

Midodrine was started, with dosage of 15 mg/day.

Follow-up: after 1 week of midodrine, patient reported a significant improvement of exercise tolerance NYHA 1, obstruction decrease to 30 mmHg at rest and 50 mmHg during exercise.

No adverse event was detected.

### Example 6

### Description of a Fifth Clinical Case

This 65 years old patient suffered of hypertrophic cardiomyopathy.

His septum wall was measured at 16 mm, he was treated with Verapamil (120 mg/d), and suffered from exercise short breathness (NYHA 2) and chest pain.

Echocardiography revealed a rest obstruction at 60 mmHg increasing to 90 mmHg during walking test and 120 mmHg during recovery.

Midodrine was started with dosage of 15 mg/day.

Follow-up: after 1 week of midodrine, patient reported a significant improvement of exercises tolerance NYHA 1-2, decrease of chest pain, obstruction was still high close to 60 mmHg at rest and 70 mmHg during exercise.

Evening blood pressure was measured at 150 mmHg.

### Example 7

### Description of a Sixth Clinical Case

This 64 years old patient suffered of hypertrophic cardiomyopathy (maron 1). His septum wall was measured at 15 mm. He has a treatment based on Carvedilol (5 mg/day), and suffered from exercise short breathness (NYHA 2). Patient also had orthostatic hypotension and chest pain.

Echocardiography revealed a rest obstruction at 40 mmHg increasing to 100 mmHg during walking test.

Midodrine started with dosage of 15 mg/day.

Follow-up: after 1 week of midodrine, patient reported a significant improvement of exercise tolerance NYHA 1, disappearance of chest pain, obstruction was up to 50 mmHg at rest and 80 mmHg during exercise.

### Example 8

### Description of Veterinary Cases

Beagle dogs and European wildcats suffering hypertrophic cardiomyopathy are treated with midodrine hydrochloride as follows:

beagle dogs and European wildcats: 0.03 mg/kg/day, 0.1 mg/kg/day, 0.3 mg/kg/day or 1.0 mg/kg/day, intravenously,

beagle dogs and European wildcats: 3 mg/kg/day, intradermally. Follow-up: after 1 week of midodrine, animals exercise tolerance is tested.

### REFERENCE LIST

1. Maron B J.: “Contemporary insights and strategies for risk stratification and prevention of sudden death in hypertrophic cardiomyopathy”, Circulation 2010; 121:445-56.
2. Wigle E D, Sasson Z, Henderson M A, et al.: “Hypertrophic cardiomyopathy. The importance of the site and the extent of hypertrophy. A review', Prog Cardiovasc Dis 1985; 28:1-83.
3. Maron M S, Olivotto I, Zenovich A G, et al. Hypertrophic cardiomyopathy is predominantly a disease of left ventricular outflow tract obstruction. Circulation 2006; 114:2232-9.
4. Shah J S, Esteban M T, Thaman R, et al. Prevalence of exercise-induced left ventricular outflow tract obstruction in symptomatic patients with non-obstructive hypertrophic cardiomyopathy. Heart 2008; 94:1288-94.
5. Joshi S, Patel U K, Yao S S, et al. Standing and exercise Doppler echocardiography in obstructive hypertrophic cardiomyopathy: the range of gradients with upright activity. J Am Soc Echocardiogr 2010; 24:75-82.
6. Argulian E, Chaudhry F A. Stress testing in patients with hypertrophic cardiomyopathy. Prog Cardiovasc Dis 2012; 54:477-82.
7. Maron B J, Gottdiener J S, Roberts W C, Henry W L, Savage D D, Epstein S E. Left ventricular outflow tract obstruction due to systolic anterior motion of the anterior mitral leaflet in patients with concentric left ventricular hypertrophy. Circulation 1978; 57:527-33.
8. Maron B J, Harding A M, Spirito P, Roberts W C, Waller B F. Systolic anterior motion of the posterior mitral leaflet: a previously unrecognized cause of dynamic subaortic obstruction in patients with hypertrophic cardiomyopathy. Circulation 1983; 68:282-93.
9. Gersh B J, Maron B J, Bonow R O, et al. 2011 ACCF/AHA guideline for the diagnosis and treatment of hypertrophic cardiomyopathy: executive summary. A report of the American College of Cardiology Foundation/American Heart Association Task Force on Practice Guidelines. J Am Coll Cardiol 2011; 58:2703-38.
10. Cavalcante J L, Barboza J S, Lever H M. Diversity of mitral valve abnormalities in obstructive hypertrophic cardiomyopathy. Prog Cardiovasc Dis 2012; 54:517-22.
11. Levine R A, Vlahakes G J, Lefebvre X, et al. Papillary muscle displacement causes systolic anterior motion of the mitral valve. Experimental validation and insights into the mechanism of subaortic obstruction. Circulation 1995; 91:1189-95.
12. Goodman and Gilman's the pharmacological basis of therapeutics, eleventh edition, chapter 10, pp 271-295,2006.
13. Lang R M, Bierig M, Devereux R B, et al. Recommendations for chamber quantification: a report from the American Society of Echocardiography's Guidelines and Standards Committee and the Chamber Quantification Writing Group. J Am Soc Echocardiogr 2005; 18: 1440-63.
14. Gottdiener J S, Bednarz J, Devereux R, et al. American Society of Echocardiography recommendations for use of echocardiography in clinical trials. J Am Soc Echocardiogr 2004; 17:1086-119.
15. Quinones M A, Douglas P S, Foster E, et al. ACC/AHA clinical competence statement on echocardiography: a report of the American College of Cardiology/American Heart Association/American College of Physicians-American Society of Internal Medicine Task Force on Clinical Competence. J Am Coll Cardiol 2003; 41:687-708.

1. A substance selected among midodrine, a pharmaceutical salt, a prodrug and an active metabolite thereof, for use in the treatment of obstructive cardiopathy.

2. The substance for use according to claim 1, wherein the pharmaceutical salt is hydrochloride.

3. The substance for use according to claim 1, wherein the active metabolite is desglymidodrine.

4. The substance for use according to claim 1, wherein the obstructive cardiopathy is selected among hypertrophic cardiomyopathy, hypertensive cardiopathy, a malposition of papillary muscles, and a situation with primary hyperkinesia of left ventricle in non hypertrophic cardiomyopathy.

5. The substance for use according to claim 1, wherein the treatment includes an administration of a dose of the substance comprised of from 6 mg to 45 mg per day.

6. The substance for use according to claim 1, wherein the treatment includes an administration of the substance which is realized from one to eight times per 24 hours.

7. The substance for use according to claim 1, wherein the treatment includes the improvement of at least one symptom of the obstructive cardiopathy selected among exercise breathlessness, exercise chest pain, exercise discomfort and exercise dizziness.

8. The substance for use according to claim 1, wherein the treatment includes an oral administration of the substance.

9. The substance for use according to claim 8, wherein the oral administration is a buccal or a sublingual administration.

10. The substance for use according to claim 8, wherein the substance is in a form selected from the group including a liquid formulation, an oral effervescent dosage form, an oral powder, a pill, a multiparticule system, an orodispersible dosage form, a solution, a syrup, a suspension, an emulsion and oral drops.

11. The substance for use according to claim 1, wherein the treatment includes a parenteral administration of the substance.

12. The substance for use according to claim 11, wherein the parenteral administration is selected from the group including intravenous administration, intramuscular administration and subcutaneous administration.

13. The substance for use according to claim 12, wherein the substance is in the form of an injectable solution.

14. The substance for use according to claim 1, for an administration to a human or an animal selected among from the group including felids, canids, cervids, equids, mustelids, procyonids, viverrids and ursids.

15. The substance for use according to claim 1, together with at least one another active compound selected among the group including beta-blockers, calcium channel blockers, antiarrhythmic agents, and anticoagulant agents.

16. The substance for use according to claim 2, wherein the obstructive cardiopathy is selected among hypertrophic cardiomyopathy, hypertensive cardiopathy, a malposition of papillary muscles, and a situation with primary hyperkinesia of left ventricle in non hypertrophic cardiomyopathy.

17. The substance for use according to claim 3, wherein the obstructive cardiopathy is selected among hypertrophic cardiomyopathy, hypertensive cardiopathy, a malposition of papillary muscles, and a situation with primary hyperkinesia of left ventricle in non hypertrophic cardiomyopathy.

18. The substance for use according to claim 2, wherein the treatment includes an administration of a dose of the substance comprised of from 6 mg to 45 mg per day.

19. The substance for use according to claim 3, wherein the treatment includes an administration of a dose of the substance comprised of from 6 mg to 45 mg per day.

20. The substance for use according to claim 4, wherein the treatment includes an administration of a dose of the substance comprised of from 6 mg to 45 mg per day.
